(19) United States
(12) Patent Application Publication
(10) Pub. No.: US 2009/0233338 A1
(43) Pub. Date: Sep. 17, 2009

(54) PROCESS FOR RECOVERY OF POLYHYDROXYALKANOATES FROM BIOMASS

(76) Inventor: Lewis G. Jacobs, Landrum, SC (US)

Correspondence Address: K&L GATES LLP;HENRY W. OLIVER BUILDING, 535 SMITHFIELD STREET, PITTSBURGH, PA, 15222, US

(21) Appl. No.: 12/400,423

(22) Filed: Mar. 9, 2009

Related U.S. Application Data
(60) Provisional application No. 61/035,838, filed on Mar. 12, 2008.

(51) Int. Cl. C12P 7/62; C12M 1/00

(52) U.S. Cl. 435/135; 435/306.1

(57) ABSTRACT

Systems and processes for the recovery of polyhydroxyalkanoates (“PHA”) from biomass are disclosed. In certain embodiments, the process includes the steps of extracting at least a portion of PHA from a disrupted quantity of PHA-containing biomass with an acetin solvent to produce a biomass phase (including residual biomass) and a PHA phase including at least a portion of the PHA from the biomass. The biomass phase and the PHA phase are separated, and at least a portion of PHA may be isolated from the PHA phase. Systems and processes including multiple disruption and/or extraction steps also are disclosed.

### CROSS REFERENCE TO RELATED APPLICATIONS

The application claims the priority benefit of U.S. Provisional Patent Application 61/035,838, filed Mar. 12, 2008, the disclosure of the entirety of which is incorporated by this reference.

### BACKGROUND

Systems and processes for the recovery of polyhydroxyalkanoates from biomass utilizing acetin-based solvents are disclosed.

Polyhydroxyalkanoates (“PHAs”) are a family of polyesters of various hydroxycarboxylic acids. Many common PHAs are polyesters of 2, 3, 4, 5, 6, 7 and 8-carbon hydroxycarboxylic acids and include, for example, polyglycolic acid (“PGA”), polylactic acid (“PLA”), polycaprolactone (“PCL”), polyhydroxypropanoate (“PHPp”), poly(3-hydroxybutyrate) (“P3HB”), poly(4-hydroxybutyrate) (“P4HB”), polyhydroxyvalerate (“PHV”), polyhydroxypentanoate (“PHPt”), polyhydroxyhexanoate (“PHHx”), polyhydroxyheptanoate, (“PHHp”), polyhydroxyoctanoate (“PHO”), and co-polymers (e.g., alternating, periodic, random, statistical, and block co-polymers) thereof.

As used herein, the term polyhydroxyalkanoate (“PHA”) refers to any polymer having the general structure:

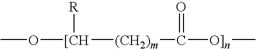

where m=0, 1, 2, . . . , 20; n can range from 2 to several thousand; and R is H or straight-chain or branched alkyl. As used herein, “PHA” further includes branched and unbranched PHA polymers, substituted and un-substituted PHA polymers, short-chain-length, medium-chain-length and long-chain-length PHAs, and PHA derivatives. In addition, as used herein, “PHA” also refers to blends, co-polymers, and mixtures of PHAs as defined herein.

PHAs are generally divided into two groups based on the number of constituent carbons atoms in their respective monomer units. Short-chain-length (“SCL”) PHAs include monomers of 3-5 carbons atoms (e.g., P3HB, P4HB and PHV). Medium-chain-length (“MCL”) PHAs include monomers of 6-14 carbons atoms (e.g., PHHX, PHHP and PHO). PHAs having more than 14 carbon atoms may be classified as long-chain length (“LCL”) PHAs. Generally, SCL PHAs are stiff and brittle with a relatively high degree of crystallinity, and MCL PHAs are flexible, have a relatively low degree of crystallinity, relatively low tensile strength, and relatively low melting point. Depending on the composition of a given PHA, the material properties of the polymeric material may be modulated. PHA materials having particular material properties may be readily produced.

Accordingly, PHAs may be engineered to possess material properties that closely mimic the material properties of petroleum-based polymers and plastics. Table 1 presents a comparison of the physical properties of various PHA compositions and conventional plastics. P3HB is highly crystalline, brittle and stiff. However, the introduction of different hydroxyalkanoate co-monomers, such as, for example, hydroxyvalerate or 3-hydroxyhexanoate, into the polymer chain substantially modulates the material properties. Copolymers of 3HB and HV generally have lower melting temperatures and lower crystallinities than P3HB, are less brittle, and tougher. P4HB is a strong, malleable and elastic thermoplastic material.

TABLE 1
Comparison of the physical properties of various PHAs and plastics.

| | | | Tensile strength | Elongation at |
|---|---|---|---|---|
| Material | Tₘ (° C.) | T_{g} (° C.) | (MPa) | break (%) |
| P3HB | 177 | 4 | 40-43 | 5-6 |
| P4HB | 60 | −50 | 104 | 100 |
| P(3HB-co-10% | 150 | NA | 25 | 20 |
| HV) | | | | |
| P(3HB-co-20% | 135 | NA | 20 | 100 |
| HV) | | | | |
| P(3HB-co-10% | 127 | −1 | 21 | 400 |
| 3HHx) | | | | |
| P(3HB-co-17% | 120 | −2 | 20 | 850 |
| 3HHx) | | | | |
| Polypropylene | 170 | −20-0 | 34 | 400 |
| Polystyrene | 110 | 85-95 | 50 | 30 |
| Polyethylene | 130 | NA | 22 | 750 |
| (HDPE) | | | | |
| Polyethylene | 115 | NA | 10 | 400 |
| (LDPE) | | | | |

P3HB is highly crystalline, brittle and stiff. However, the introduction of different hydroxyalkanoate co-monomers, such as, for example, hydroxyvalerate or 3-hydroxyhexanoate, into the polymer chain substantially modulates the material properties. Copolymers of 3HB and HV generally have lower melting temperatures and lower crystallinities than P3HB, are less brittle, and tougher. P4HB is a strong, malleable and elastic thermoplastic material.

PHAs are generally biodegradable materials. PHAs have the ability to degrade under both aerobic and anaerobic conditions. PHAs may also be degraded by hydrolytic, thermal and/or enzymatic means. PHAs may be readily degraded in both external environmental and internal biological systems. The rate of biodegradation of PHAs depends on environmental/biological conditions such as temperature, moisture, pH and chemical composition. Biodegradation of PHAs may also depend on material properties of the polymer such as monomer composition, crystallinity, functionalization and macrostructure. Control of the physical and chemical properties of PHAs allows the biodegradability of a given PHA composition to be engineered to particular specifications.

PHAs may be synthesized naturally by a wide variety of bacteria, for example, through fermentation of sugars, lipids, alkanes, alkenes and alkanoic acids. Both Gram positive and Gram negative bacteria, such as, for example, Alcaligenes, Pseudomonas, Bacillus, Ralstonia, Aeromonas, Rhodobacter, and certain Archaea (such as Halobacteria like Haloferax sulfurifontis) are able to naturally synthesize PHAs. In addition, cyanobacteria and other photosynthetic microbes naturally accumulate PHAs by photosynthesis under photoautotrophic or mixotrophic growth conditions, and halophiles such as Halomonas boliviensis produce PHAs under various growth conditions. PHAs may exist as discrete insoluble cytoplasmic granules or inclusions in PHA-producing microbes, where they may function as energy storage compounds. PHAs may accumulate to up to 90% dry cell weight in prokaryotic cells. The PHA granules can accumulate in response to nutrient limitation and serve as carbon and energy reserve materials.

Industrial production of PHAs from bacterial sources on an efficient scale has been demonstrated. For example, a strain of Alcaligenes latus grown in a mineral medium using sucrose as a carbon source produces up to 90% P3HB by dry cell weight. Industrial production of poly(3HB-co-3HHx) by Aeromonas hydrophilia has also been accomplished. In addition, transgenic microorganisms, such as, for example, a recombinant Escherichia coli K12 strain have been used to produce an elastomeric blend of P3HB and PHO that has been approved by the FDA for production of food additives. In all of these commercial-scale operations, the PHAs are produced in microbial fermentations.

PHA production has also been demonstrated in transgenic plants, for example, plants containing a transgenic PHA synthase gene. For example, P3HB and P(3HB-co-HV) have been produced in various species of Arabidopsis and in Brassica rapa. PHA production has been further investigated in other species, including alfalfa, corn, cotton, maize, potato, rapeseed, tobacco and sugar cane. In addition, the PHA metabolic pathway has been successfully transferred into Panicum virgatum (switchgrass).

PHAs present a promising “green” bioplastic alternative to conventional petroleum-based plastics given (1) the ability to engineer PHAs having specific material properties that compare to petroleum-based plastics; (2) the ability to engineer the biodegradability of PHAs; and (3) the renewable and sustainable sources of PHA production. Accordingly, PHAs have found uses in numerous commercial applications including, for example, biodegradable packaging, coatings and laminates; medical devices (such as sutures, suture fasteners, rivets, tacks, screws, bone plates, orthopedic pins and dowels, bone grafts, spinal fusion cages, surgical mesh, tissue repair patches, adhesion barriers, wound dressings, meniscus repair devices, nerve conduits, pericardial patches, artery and vein augments, stents, vascular grafts, heart valves, ligament and tendon grafts, and ocular implants, for example); tissue engineering applications (such as hydroxyapatite/PHA composites for bone tissue engineering and implant scaffolds for cellularization and tissue regeneration); drug delivery vehicles (such as carriers, excipients, tablet coatings, dusting powders, microspheres, drug-eluting implants, and other controlled biodegradable drug delivery vehicles); agricultural films and coatings (e.g., for fertilizer, herbicide and/or insecticide delivery); nanocomposites and conventional polymer blends. (See Philip et al., Journal of Chemical Technology and Biotechnology, 82:233-247 (2007) and Chen et al., Biomaterials, 26:6565-6578 (2005), both of which are incorporated in their entirety by reference herein).

The extraction and recovery of PHAs from biomass is therefore important for the renewable and sustainable production of PHAs. Furthermore, it is desirable that the recovery processes do not utilize extraction solvents and/or other processing chemicals that are hazardous or toxic to humans. This is particularly important, for example, in food, beverage, medical device, tissue engineering and drug delivery applications, where non-toxicity and biocompatibility may be important. Accordingly, there is a need for the development of methods for the commercial scale processing, recovery and purification of PHAs from biomass. It is therefore an object of the present invention to provide systems and processes for the recovery of PHA from biomass.

### SUMMARY

The various embodiments are directed to systems and processes for the recovery of PHA from biomass. In one general aspect, a process for recovery of PHA from biomass includes the step of extracting at least a portion of PHA from PHA-containing biomass with an acetin solvent. The extraction produces a biomass phase and a PHA phase. The biomass phase includes residual biomass. The PHA phase includes at least a portion of the PHA from the biomass. The biomass phase and the PHA phase are separated.

In another general aspect, a process for the recovery of PHA from PHA-containing biomass includes the step of disrupting one or more cell in the biomass. At least a portion of the PHA is extracted from the biomass with a first amount of an acetin solvent. A first PHA phase and a first biomass phase are produced. The first PHA phase includes at least a portion of the PHA. The first biomass phase and the first PHA phase are separated.

In yet another general aspect, a process for the recovery of PHA from PHA-containing biomass includes the step of disrupting one or more cells in the biomass. A first amount of an acetin solvent at a temperature from about 25° C. to about 200° C. is used to extract at least a portion of the PHA from the biomass. A first biomass phase and a first PHA phase are produced. From about 0.1% to about 20% of the PHA phase by weight is PHA. The acetin solvent is one of triacetin, 1,2-diacetin, 1,3-diacetin, 1-monoacetin, 2-monoacetin, and mixtures of any two or more thereof. The first PHA phase is separated from the first biomass phase. At least a portion of the PHA in the first PHA phase is isolated from the first PHA phase.

### BRIEF DESCRIPTION OF THE DRAWINGS

The various embodiments may be understood by reference to the following description, taken with the accompanying drawings as follows.

FIG. 1 is a process diagram that illustrates a system and process for recovery of PHA from biomass where an amount of an acetin solvent is mixed with biomass prior to disruption of the biomass.

FIG. 2 is a process diagram that illustrates a system and process for recovery of PHA from biomass where an amount of an acetin solvent is mixed with biomass subsequent to disruption of the biomass.

FIG. 3 is a process diagram that illustrates a system and process for recovery of PHA from biomass having two disruption steps, where an amount of an acetin solvent is mixed with biomass between the two disruption steps.

FIG. 4 is a process diagram that illustrates a system and process for recovery of PHA from biomass having two disruption steps and two separation steps, where a first amount of an acetin solvent is mixed with the biomass prior to the first disruption step and a second amount of an acetin solvent is mixed with the biomass prior to the second disruption step.

FIG. 5 is a process diagram that illustrates a system and process for recovery of PHA from biomass having two disruption steps and two separation steps, where a first amount of an acetin solvent is mixed with the biomass subsequent to the first disruption step and a second amount of an acetin solvent is mixed with the biomass subsequent to the second disruption step.

FIG. 6 is a process diagram that illustrates a system and process for recovery of PHA from biomass analogous to the system and process illustrated in FIG. 5 except that the first PHA phase and the second PHA phase are mixed to form a combined PHA phase.

FIG. 7 is a process diagram that illustrates a system and process for recovery of PHA from biomass having three disruption steps and three separation steps, where a first amount of an acetin solvent is mixed with the biomass prior to the first disruption step, a second amount of an acetin solvent is mixed with the biomass prior to the second disruption step, and a third amount of an acetin solvent is mixed with the biomass prior to the third disruption step.

FIG. 8 is a process diagram that illustrates a system and process for recovery of PHA from biomass analogous to the system and process illustrated in FIG. 8, except that the first PHA phase, the second PHA phase and the third PHA phase are mixed together to form a combined PHA phase.

FIG. 9 is a process diagram that illustrates a system and process for recovery of PHA from biomass analogous to the system and process illustrated in FIG. 9, except that only the first PHA phase and the second PHA phase are mixed together to form a combined PHA phase.

FIG. 10 is a process diagram that illustrates a system and process for recovery of PHA from biomass having three disruption steps and two separation steps, Where a first amount of an acetin solvent is mixed with the biomass between the first disruption step and the second disruption step, and a second amount of an acetin solvent is mixed with the biomass prior to the third disruption step.

FIG. 11 is a process diagram that illustrates a system and process for recovery of PHA from biomass analogous to the system and process illustrated in FIG. 10, except that the first PHA phase and the second PHA phase are mixed together to form a combined PHA phase.

FIG. 12 is a process diagram that illustrates a system and process for recovery of PHA from biomass having a disruption step, a separation step, and an isolation/purification step.

FIG. 13 is a process diagram that illustrates a system and process for recovery of PHA from biomass having a disruption step, a separation step, an isolation/purification step, and a recycle step.

FIG. 14 is a process diagram that illustrates an embodiment of an isolation/purification unit including a wiped film evaporation unit and a short path distillation unit.

FIG. 15 is a schematic that illustrates an embodiment of an isolation/purification unit including a horizontal film rotary evaporation unit.

### DETAILED DESCRIPTION

It is to be understood that certain descriptions of the present invention have been simplified to illustrate only those elements and limitations that are relevant to a clear understanding of the present invention, while eliminating, for purposes of clarity, other elements. Those of ordinary skill in the art, upon considering the present description of the invention, will recognize that other elements and/or limitations may be desirable in order to implement the present invention. However, because such other elements and/or limitations may be readily ascertained by one of ordinary skill upon considering the present description of the invention, and are not necessary for a complete understanding of the present invention, a discussion of such elements and limitations is not provided herein. As such, it is to be understood that the description set forth herein is merely exemplary to the present invention and is not intended to limit the scope of the claims.

Other than in the examples herein, or unless otherwise expressly specified, all of the numerical ranges, amounts, values, and percentages, such as those for amounts of materials, elemental contents, times and temperatures of reaction, ratios of amounts, and others, in the following portion of the specification and attached claims may be read as if prefaced by the word “about,” even though the term “about” may not expressly appear with the value, amount, or range. Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the deviation found in its underlying respective testing measurements. Furthermore, when numerical ranges are set forth herein, these ranges are inclusive of the recited range end points (i.e., end points may be used). Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

All patents, publications, or other disclosure material referenced herein are incorporated by reference in their entirety. However, any patent, publication, or other disclosure material, in whole or in part, that is incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The articles “a,” “an,” and “the” are used herein to refer to one or more than one (i.e., to at least one) of the grammatical objects of the article. By way of example, “a component” means one or more components, and thus, possibly, more than one component is contemplated and may be employed or used.

The present disclosure describes several different features and aspects of the various exemplary embodiments provided herein. It is understood, however, that the present disclosure embraces numerous alternative embodiments, which may be accomplished by combining any of the different features, aspects, and embodiments described herein in any combination that one of ordinary skill in the art would find useful.

The various embodiments relate, in general, to systems and processes for recovery of PHA from biomass. According to a non-limiting embodiment, the systems and processes described herein include extracting at least a portion of PHA from the biomass with an acetin solvent to provide a biomass phase comprising residual biomass and a PHA phase comprising at least a portion of the PHA from the biomass, and separating the PHA phase from the biomass phase.

In certain non-limiting embodiments of the systems and processes described herein, the acetin solvent may be triacetin, 1,2-diacetin, 1,3-diacetin, 1-monoacetin, 2-monoacetin, and mixtures of any two or more thereof. According to a non-limiting embodiment of the systems and processes described herein, extracting at least a portion of PHA from biomass with an acetin solvent may comprise contacting the biomass and the acetin solvent, where the acetin solvent is at a temperature from about 25° C. to about 200° C. According to another non-limiting embodiment of the systems and processes described herein, the biomass may be one or more of a microorganism capable of producing the PHA, an agricultural biomass capable of producing the PHA, and combinations of any two or more thereof. Moreover, the biomass may be a genetically modified microorganism, a genetically modified plant source, a corn stover, a switchgrass, a sugarcane, an oil seed, and combinations of any two or more thereof.

According to another non-limiting embodiment, the systems and processes described herein include the step of disrupting one or more cells in the biomass. According to yet another non-limiting embodiment, the disrupting of one or more cells in the biomass may comprise at least one of pulverizing one or more cells of the biomass and micropulverizing one or more cells of the biomass. According to another non-limiting embodiment, the disrupting may be performed in a disrupting device. Suitable disrupting devices include, but are not limited to, a ball mill, a pulverizing mill, a hammer mill, a cell grinder, an inline emulsifier, a high shear emulsifier, a sonic agitator, an ultrasonicator, and combinations of any two or more of the foregoing devices. According to another non-limiting embodiment, the separation of the PHA phase from the biomass phase may be performed by an operation such as, for example, settling, centrifuging, filtering, decanting, and combinations of any two or more thereof.

According to another non-limiting embodiment, the systems and processes described herein may include the step of isolating at least a portion of the PHA from the PHA phase. The isolation may be performed, for example, by cooling the PHA phase, adding a co-solvent to the PHA phase, and/or removing at least a portion of the acetin solvent. According to another non-limiting embodiment, the systems and processes described herein may include the step of purifying the isolated PHA. The purification may be performed by an operation such as, for example, membrane separation, ultra filtration, vacuum drying, short path stripping, wiped film evaporation, wiped film stripping, horizontal film rotary evaporation, horizontal film rotary stripping, centrifuging, supercritical extraction, and combinations of any two or more thereof.

According to another non-limiting embodiment, the systems and processes described herein may include disrupting one or more cells in a quantity of biomass comprising PHA, extracting at least a portion of the PHA from the biomass with a first amount of an acetin solvent to provide a first biomass phase and a first PHA phase comprising the at least a portion of the PHA, and separating the first PHA phase from the first biomass phase. According to another non-limiting embodiment, the systems and processes described herein may include the steps of contacting the first biomass phase with a second amount of an acetin solvent, disrupting one or more cells in the first biomass phase to provide a second biomass phase and a second PHA phase comprising at least a second portion of PHA, and separating the second PHA phase from the second biomass phase.

According to another non-limiting embodiment, the systems and processes described herein may include the steps of combining the first PHA phase with the second PHA phase to provide a combined PHA phase, and isolating PHA from the combined PHA phase. In a further aspect, the biomass and the first amount of acetin solvent may be mixed prior to disrupting one or more cells of the biomass. According to another non-limiting embodiment, the systems and processes described herein may include the steps of contacting the second biomass phase with a third amount of an acetin solvent, disrupting one or more cells in the second biomass phase to provide a third biomass phase and a third PHA phase comprising at least a third portion of PHA, separating the third PHA phase from the third biomass phase, and isolating the at least a third portion of the PHA from the third PHA phase.

Definitions

As defined herein, the terms “polyhydroxyalkanoate”, “PHA”, “PHA polymer”, and “PHA material” includes oligomers, polymers, and materials comprising oligomers and polymers having the general structure:

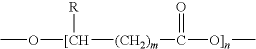

where m=0, 1, 2, . . . , 20; n can range from 2 to several thousand; and R is H or straight-chain or branched alkyl. As used herein, the terms “polyhydroxyalkanoate”, “PHA”, “PHA polymer”, and “PHA material” further include branched and un-branched PHA polymers, substituted and un-substituted PHA polymers, short-chain-length, medium-chain-length and long-chain-length PHA, and PHA derivatives.

In addition, as used herein, the terms “polyhydroxyalkanoate”, “PHA”, “PHA polymer”, and “PHA material” includes blends, co-polymers, mixtures, and materials comprising PHA as defined herein. Furthermore, the terms “polyhydroxyalkanoate”, “PHA”, “PHA polymer”, and “PHA material” refer to any PHA-based materials initially present in the biomass prior to processing, and products formed during processing such as products formed from degradation. This includes processes occurring within the biomass or derivative products formed by treatment of the biomass with physical, chemical or biological agents to cause a chemical transformation.

In various embodiments, the systems and processes described herein are useful for recovering PHA from biomass. In various embodiments, the systems and processes described herein are useful for recovering PHA homopolymers from biomass. Additionally, co-polymers including at least two different monomers of a hydroxy acid may be recovered from biomass. PHA including oligomers, polymers and derivatives of 2-, 3-, 4-, 5-, 6-, 7- and 8-carbon hydroxycarboxylic acids may be recovered from biomass, for example. Exemplary recovery products include, for example, PLA, PHPp, P3HB, P4HB, PHV, P3HB-co-PHV, PHPt, PHHx, PHHp, PHO, and co-polymers (e.g., alternating, periodic, random, statistical, and block co-polymers), blends, and mixtures of any two or more thereof.

The PHA polymers may also contain or be modified to include other non-hydroxycarboxylic acid units, such as, for example, long chain fatty acids, amino acids, carbohydrates, phosphorus and sulfur containing compounds, and/or sugar alcohols such as glycerol. PHA materials which can be recovered include derivatives, for example, formed upon physical, chemical, and/or biochemical treatment of biomass or by processes within the biomass. Derivatives may include, for example, hydroxycarboxylic acid dimers, trimers, linear and cyclic oligomers and lactones. Other non-limiting examples of PHA derivative products that can be isolated from the biomass include esters, diols, unsaturated compounds, aldehydes, carboxylic acids, alcohols, lactones, linear esters, amides and thioesters of PHAs.

As used herein, the term “biomass” includes any organic material, for example, plant, animal, fungal, bacterial, or algal based. Preferably, in certain embodiments, the biomass may be-available on a renewable or recurring basis. Organic material may include, but is not limited to, plant biomass such as dedicated energy crops; agricultural crops; natural vegetation; trees; food, feed and fiber crops residues; and other non-fossil organic materials. Biomass may be further defined to include primary biomass (produced by agriculture and forestry), secondary biomass (residues produced as a result of harvesting and processing of primary biomass), and tertiary biomass (post-consumer residue streams).

In various embodiments, PHA materials may be isolated from plant biomass derived from plants such as, for example, soybean, cotton, coconuts, groundnuts, rapeseed, sunflower, olive, palm, sesame seed, linseed, castor, safflower seed, tobacco, and potato. Plant biomass may include plant components such as seeds, leaves, stalks, flowers, fruits, roots and stems. Exemplary agricultural biomass includes, but is not limited to, corn stover, switchgrass, sugarcane, various varieties of oil seed plants, and combinations of any two or more thereof. In certain embodiments, the biomass may be an oil seed plant such as rapeseed, sunflower, safflower, linseed and/or soybean. In embodiments where the biomass comprises oil seed plants, both the plant oil and the PHAs may be recovered from the biomass.

In various embodiments, the biomass may be derived from transgenic plants. As used herein, the term “transgenic plants” includes plants that have been modified to include genetic material from at least one different species of organism and includes plants that have been propagated from genetically modified plants. Transgenic plants for production of PHAs may be obtained using methods available in the art. PHA polymers typically can be produced using propagation of transgenic plants at a price and on a scale that is competitive with petrochemical derived plastics. Transgenic plant derived PHA polymers or their derivatives can be processed and recovered from transgenic plant biomass in commercially useful forms. The location of the PHA in the plant crop (e.g., in the plant seeds, leaves, stalks, flowers, fruits, roots, stems, or combinations of any two or more thereof) can be varied to maximize the yield of PHA from the plant.

In other embodiments, PHA materials may be isolated from bacterial biomass. Bacterial biomass may include natural PHA-producing bacteria, transgenic PHA-producing bacteria or mixtures thereof. In addition, bacterial biomass may include mixtures of different varieties of PHA-producing bacteria, for example, mixed cultures of Escherichia coli and Aeromonas hydrophilia. Furthermore, biomass may include mixtures of plant biomass, bacterial biomass, and/or any other type of PHA-containing biomass.

Various types of PHA-containing biomass may require disruption in order to release the PHA from the biomass. For example, in PHA producing bacteria, the PHA may be found as discrete insoluble cytoplasmic granules or inclusions integrated in the cellular material. The PHA in the bacteria may be protected from dissolution in solvents by at least the cell wall of the microbes. In PHA producing plants, the cell walls also may prevent dissolution of intracellular PHA in extraction solvents. Accordingly, it may be necessary to disrupt the biomass, or components within the biomass (such as, for example, cells within the biomass), in order to provide increased solvent accessibility to the intracellular PHA.

As used herein, the terms “extraction”, “extract”, “extracting”, and “extracted” includes the process of contacting a material with a solvent where the material comprises at least one component of interest, and the component is at least partially soluble in the solvent such that at least a portion of the component is removed from the material by the solvent. For example, the term “extraction” and grammatical variations thereof include, but are not limited to, the process of contacting a biomass comprising PHA with an amount of an acetin solvent such that at least a portion of the PHA from the biomass dissolves in the acetin solvent.

As used herein, the terms “disruption”, “disrupt”, “disrupting”, and “disrupted” includes the operation of rupturing, fracturing, or otherwise breaking of biomass, including, for example, cellular and intracellular structures within the biomass. In certain embodiments, disrupting the biomass may increase the accessibility of an extraction solvent (e.g., an acetin solvent) to the intracellular PHA. In certain embodiments, the disruption operation may function to breach the cell walls of microbiological and/or plant biomass that contains intracellular PHA. In other embodiments, the disruption operation may function to breach the structure of intracellular material, such as cytoplasmic granules or inclusions, for example. Disruption may be accomplished, for example, by mechanical means (e.g., chopping and/or grinding), physical means (e.g., ultrasonication), chemical means (e.g., acid digestion), biochemical means (e.g., enzymatic digestion), or combinations of any two or more thereof. According to a non-limiting embodiment, the disruption operation may also involve biomass particle size reduction, dehydration, pyrolysis, and other suitable pre-extraction processing steps that aid in the solubilization of the intracellular PHA.

As used herein, the term “acetin” refers to any of the five possible acetate structures formed from the esterification of glycerol (propane-1,2,3-triol) with acetic acid (ethanoic acid). The five acetins are triacetin, 1,3-diacetin, 1,2-diacetin, 1-monoacetin, and 2-monoacetin. The structures of the acetins are illustrated below.

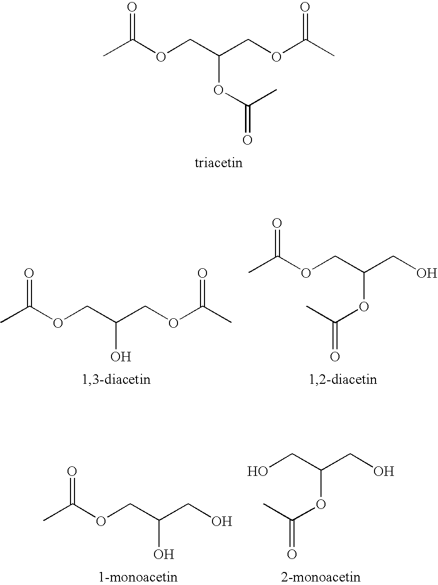

As used herein, the term “acetin” also includes mixtures of any two or more of the five acetins. As used herein, the term “acetin solvent” includes a solvent comprising at least one acetin.

Triacetin is generally regarded as safe (“GRAS”) by the FDA as a human food ingredient. The monoacetins and diacetins have been approved by the FDA for use as food additives. Therefore, all of the acetins are food grade materials. In addition, acetins are generally insoluble in aqueous media. Accordingly, acetins represent safe and biocompatible extraction solvents that may be used to recover PHAs from biomass and do not pose a toxicity hazard like many conventional extraction solvents.

In addition to acetin solvents, solvents suitable for use in various embodiments of the systems and processes described herein may include various acetates of higher sugar alcohols. For example, acetates of butane-1,2,3,4-tetrol (erythritol), pentane-1,2,3,4,5-pentanol (xylitol), and hexane-1,2,3,4,5,6-hexol (mannitol and sorbitol) may be suitable as solvents for separating PHA in the systems and processes described herein. In addition, solvents including molecules comprising sugar alcohol esters of higher chain alkyl-carboxylic acids such as, for example, propanoic acid, butanoic acid, pentanoic acid and hexanoic acid may be suitable for use in the systems and processes described herein. Additional suitable solvents include, for example, propanoic acid esters of glycerol (e.g., glyceryl monopropanoate, glyceryl dipropanoate, glyceryl tripropanoate, mixtures thereof, or mixtures of glyceryl propanoates and acetins).

Ethylene glycol (ethane-1,2-diol) is a simple sugar alcohol consisting of two hydroxyl groups and an ethyl group. Ethylene glycol based solvents (e.g., ethylene glycol diacetate) are technically capable of functioning as extraction solvents in the systems and processes described herein. However, ethylene glycol based solvents are metabolized in biological systems to ethylene glycol, which is highly toxic. Therefore, given the toxicity of ethylene glycol based solvents, such solvents are less desirable as extraction solvents for use in the systems and processes described herein. Acetins and other sugar alcohol esters are metabolized in biological systems to glycerol and other sugar alcohols, which are not toxic and are GRAS. Accordingly, the use of acetins and other sugar alcohol esters as extraction solvents for recovering PHA from biomass provides a safe, non-toxic and biocompatible solvent system.

In various non-limiting embodiments, at least a portion of PHA is extracted from biomass by mixing the biomass with an acetin solvent to form an extraction mixture. The extraction mixture comprises a PHA phase and a biomass phase. The PHA phase comprises at least a portion of the PHA extracted from the biomass and the acetin solvent. The biomass phase comprises residual biomass, which may include additional PHA. The PHA phase is separated from the biomass phase. From about 0.1% to about 20% of the polyhydroxyalkanoate phase by weight is polyhydroxyalkanoate.

In various non-limiting embodiments, the acetin solvent is mixed with or otherwise contacted with the biomass at a temperature ranging from about 25° C. to about 200° C. In various non-limiting embodiments, the acetin solvent is mixed with or otherwise contacted with the biomass at a temperature of about 50° C. to about 120° C., about 60° C. to about 110° C., about 70° C. to about 100° C., or about 80° C. to about 90° C. In various non-limiting embodiments, the same or different acetin solvents may be contacted with different biomass phases in a sequential recovery operation. Also, in various non-limiting embodiments, acetin solvents at different temperatures may be contacted with different biomass phases in a sequential recovery operation.

In various non-limiting embodiments, the biomass may be disrupted prior to contact with an acetin solvent or the disrupting of the biomass may be conducted in the presence of the acetin solvent, thereby simultaneously extracting PHA from the biomass during the disruption. The biomass phase may be further contacted with a second or subsequent amount of an acetin solvent to form a second or subsequent extraction mixture which comprises a second or subsequent biomass phase and PHA phase. The series of PHA phases (i.e., a first PHA phase, a second PHA phase, etc.) may be separately processed to obtain isolated/purified PHA or combined for further processing. In various non-limiting embodiments, the PHA phase from a given phase separation process (i.e., the first PHA phase, the second PHA phase, etc.) comprises from about 0.1% to about 20% by weight of PHA. The PHA phase may also comprise from about 1% to about 15%, from about 5% to about 10%, or from about 6% to about 8% by weight PHA.

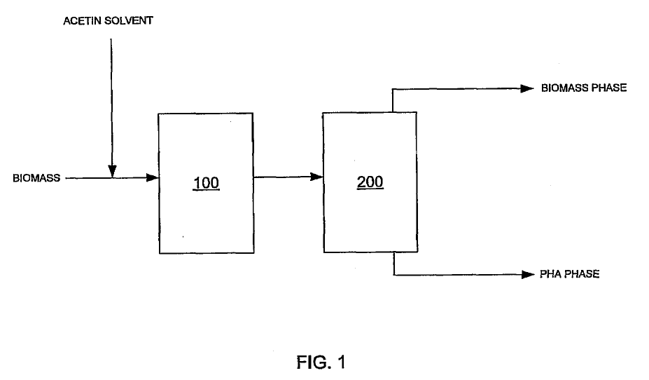

FIG. 1 is a process diagram that illustrates a non-limiting embodiment of a system and process for recovery of PHA from biomass where an amount of an acetin solvent is mixed with the biomass prior to disruption of the biomass. The biomass is mixed with an amount of an acetin solvent to form an extraction mixture. The extraction mixture is introduced to a disruption unit 100, where at least one cell in the biomass (for example a PHA-producing bacterium or plant cell, such as described herein) is disrupted. The disruption unit 100 comprises at least one cell disruption device which conducts the cell disruption operation. The cell disruption operation may be conducted according to any suitable cell disruption method including, for example, hypotonic lysis, enzymatic digestion, chemical digestion, mechanical agitation, bead agitation, sonic agitation, sonication, grinding, chopping, ball milling, pulverization milling, micropulverization, hammer milling, inline emulsification/mixing, high shear emulsification/mixing, the “cell bomb” method (i.e., cells are placed under high pressure (usually nitrogen or other inert gas up to about 25,000 psi) followed by rapid decompression causing the dissolved gas to be released, lysing the cell), rotor-stator processing, valve-type processing (e.g., French pressing and/or mechanically pumped-fluid processing), fixed-geometry fluid processing, and combinations of any two or more thereof. Cell disruption devices suitable for use in the processes and systems described herein include, for example, digestion tanks, ball mills, pulverizing mills, hammer mills, cell grinders, inline emulsifiers/mixers (e.g., available from IKA Works, Inc., Wilmington, N.C., USA), high shear emulsifiers/mixers (e.g., available from IKA Works, Inc., Wilmington, N.C., USA), sonicators, ultrasonicators, Sonolators™ (available from Sonic Corporation, Stratford, Conn., USA), and Microfluidizers™ (available from Microfluidics Corporation, Newton, Mass., USA).

The extraction mixture comprises at least a PHA phase and a biomass phase. The PHA phase comprises at least a portion of the PHA, which has been extracted from the biomass with the acetin solvent. The biomass phase comprises residual biomass. The extraction mixture exits the disruption unit 100 and is introduced to a separation unit 200, where the PHA phase is separated from the biomass phase. The separation unit 200 comprises at least one separation apparatus which conducts the separation operation. The separation operation may be conducted according to any suitable separation method known in the art including, for example, settling, sedimentation, clarification, centrifugation, filtration (gravity, vacuum, and/or pressure), decanting, and combinations of any two or more thereof. Separation apparatuses suitable for use in the processes and systems described herein include, for example, settling, sedimentation and/or clarification tanks; centrifuges; filters; and decanters. The separated biomass phase and PHA phase exit the separation unit 200 and are individually available for further processing.

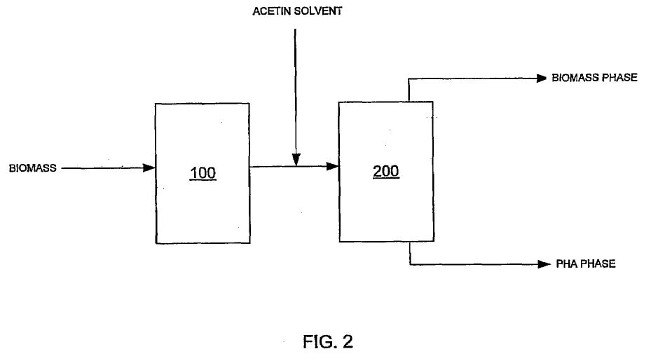

FIG. 2 is a process diagram that illustrates a non-limiting embodiment of a system and process for recovery of PHA from biomass where an acetin solvent is mixed with a quantity of biomass subsequent to disruption of the biomass. The biomass is introduced to disruption unit 100, where at least one cell in the biomass is disrupted as described herein. The disrupted biomass exits the disruption unit 100 and is mixed with an amount of an acetin solvent to form an extraction mixture. The extraction mixture comprises at least a PHA phase and a biomass phase. The extraction mixture is introduced to separation unit 200, where the PHA phase is separated from the biomass phase. The separated biomass phase and PHA phase exit the separation unit 200 and are individually available for further processing.

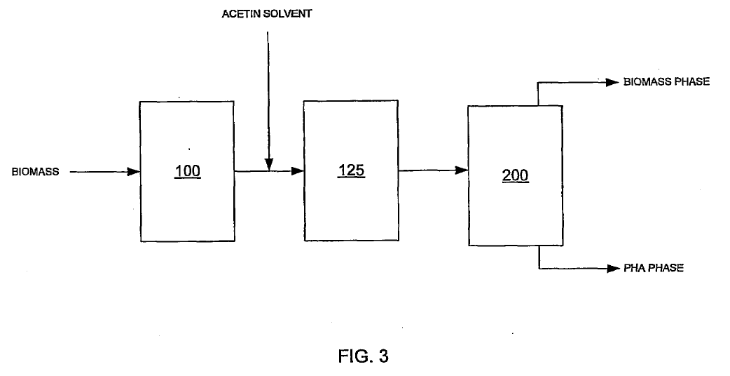

FIG. 3 is a process diagram that illustrates a non-limiting embodiment of a system and process for recovery of PHA from biomass having two disruption steps, where an amount of an acetin solvent is mixed with the biomass between the two disruption steps. The biomass is introduced to the first disruption unit 100, where at least one cell in the biomass is disrupted. The disrupted biomass exits the first disruption unit 100 and is mixed with an acetin solvent to form an extraction mixture. The extraction mixture comprises at least a PHA phase and a biomass phase. The extraction mixture is introduced to the second disruption unit 125, where the biomass in the extraction mixture is further disrupted. The extraction mixture exits the second disruption unit 125 and is introduced to separation unit 200, where the PHA phase comprising at least a portion of the PHA from the biomass is separated from the biomass phase. The separated biomass phase and PHA phase exit the separation unit 200 and are individually available for further processing. The first disruption unit 100 and the second disruption unit 125 may each comprise the same disruption device(s) or may comprise different disruption devices. The systems and process described herein are not limited in this context.

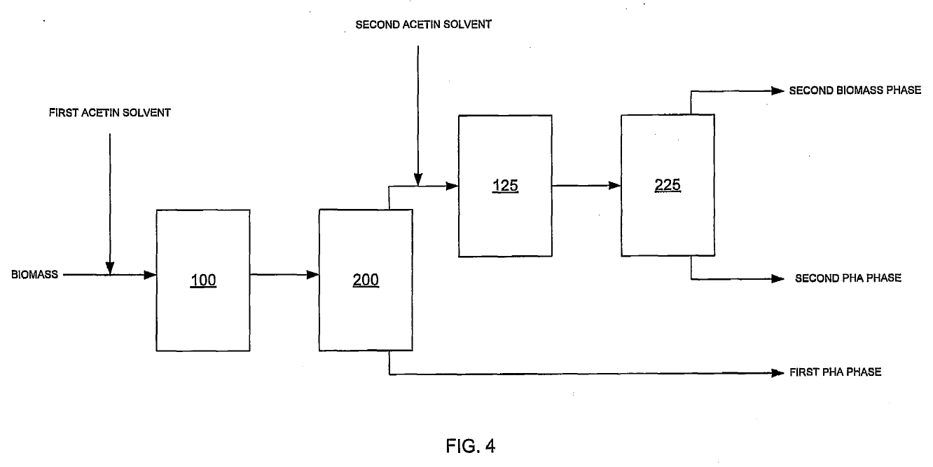

FIG. 4 is a process diagram that illustrates a non-limiting embodiment of a system and process for recovery of PHA from biomass having two disruption steps and two separation steps, where a first amount of an acetin solvent is mixed with a quantity of the biomass prior to the first disruption step and a second amount of an acetin solvent is mixed with the biomass prior to the second disruption step. The biomass is mixed with a first amount of acetin solvent to form a first extraction mixture. The first extraction mixture comprises at least a first PHA phase and a first biomass phase. The first extraction mixture is introduced to the first disruption unit 100, where at least one cell in the biomass is disrupted. The first extraction mixture exits the first disruption unit 100 and is introduced to the first separation unit 200, where the first PHA phase is separated from the first biomass phase. The separated first biomass phase and first PHA phase exit the first separation unit 200. The separated first PHA phase is available for further processing.

The separated first biomass phase is mixed with a second amount of an acetin solvent (which may be the same or different from the composition and/or amount of acetin solvent used in the first extraction step) to form a second extraction mixture. The second extraction mixture comprises a second PHA phase and a second biomass phase. The second PHA phase comprises at least a portion of the PHA remaining in the disrupted first biomass phase. The second extraction mixture is introduced to the second disruption unit 125, where the biomass is further disrupted. The second extraction mixture exits the second disruption unit 125 and is introduced to the second separation unit 225, where the second PHA phase is separated from the second biomass phase. The separated second biomass phase and second PHA phase exit the second separation unit 225 and are individually available for further processing.

In various non-limiting embodiments, the first amount of an acetin solvent and the second amount of an acetin solvent may be identical amounts of an identical solvent composition at an identical temperature. In various non-limiting embodiments, the first amount of an acetin solvent and the second amount of an acetin solvent may be different amounts. In various non-limiting embodiments, the first amount of an acetin solvent and the second amount of an acetin solvent may be at different temperatures. In various non-limiting embodiments, the first amount of an acetin solvent and the second amount of an acetin solvent may be different solvent compositions. For example, in various non-limiting embodiments the first amount of an acetin solvent may be a specific acetin or mixture of acetins and the second amount of an acetin solvent may be a different specific acetin or different mixture of acetins. The first amount of acetin solvent and the second amount of acetin solvent may be at the same or different temperatures and may be contacted with the biomass in the same or different quantities. The systems and processes described herein are not limited in this context.

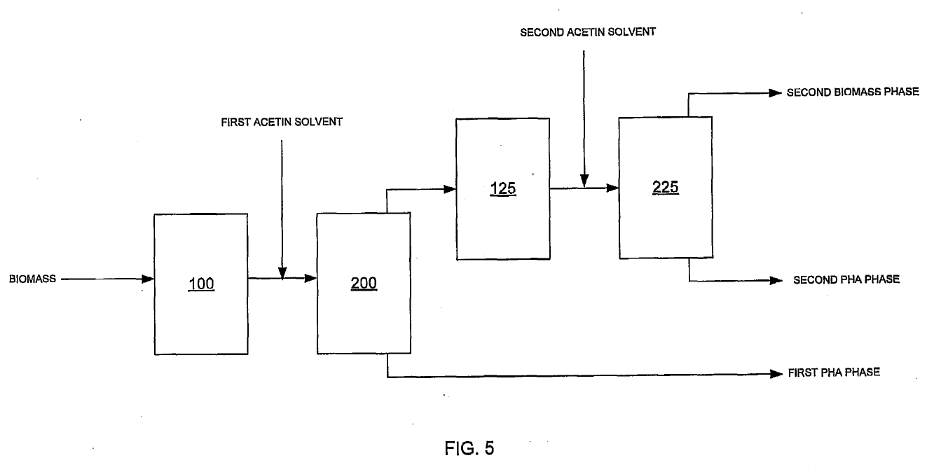

FIG. 5 is a process diagram that illustrates a non-limiting embodiment of a system and process for recovery of PHA from biomass having two disruption steps and two separation steps, where a first amount of an acetin solvent is mixed with the biomass subsequent to the first disruption step and a second amount of an acetin solvent is mixed with the biomass subsequent to the second disruption step. The biomass is introduced to the first disruption unit 100, where at least one cell in the biomass is disrupted. The biomass exits the first disruption unit 100 and is mixed with a first amount of an acetin solvent to form a first extraction mixture. The first extraction mixture comprises at least a first PHA phase and a first biomass phase. The first PHA phase comprises at least a portion of the PHA originally present in the biomass. The first extraction mixture is introduced to the first separation unit 200, where the first PHA phase is separated from the first biomass phase. The separated first biomass phase and first PHA phase exit the first separation unit 200. The separated first PHA phase is available for further processing.

The separated first biomass phase is introduced to the second disruption unit 125, where the biomass is further disrupted. The biomass exits the second disruption unit 125 and is mixed with a second amount of an acetin solvent to form a second extraction mixture. The second extraction mixture comprises at least a second PHA phase and a second biomass phase. The second PHA phase comprises at least a portion of the PHA remaining in the first biomass phase. The second extraction mixture is introduced to the second separation unit 225, where the second PHA phase is separated from the second biomass phase. The separated second biomass phase and second PHA phase exit the second separation unit 225 and are individually available for further processing.

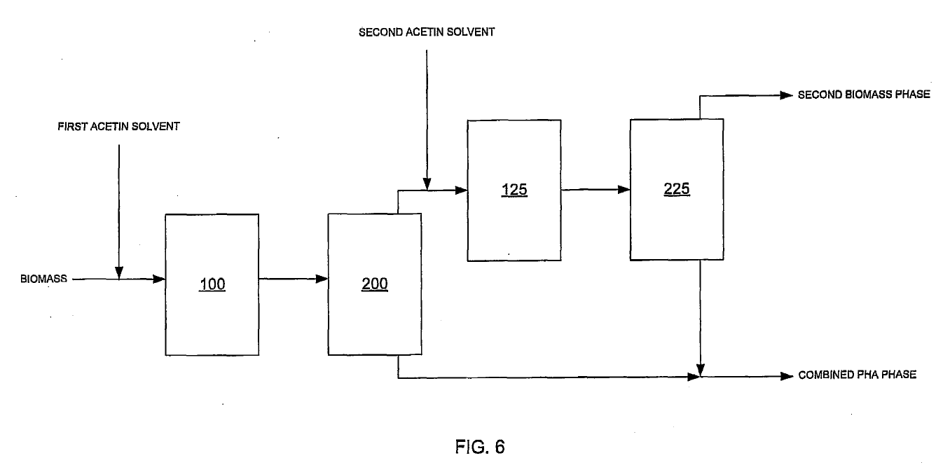

FIG. 6 is a process diagram that illustrates a non-limiting embodiment of a system and process for recovery of PHA from biomass analogous to the system and process illustrated in FIG. 4, except that the first PHA phase and the second PHA phase as described herein are mixed to form a combined PHA phase comprising at least a portion of the PHA from the biomass. The combined PHA phase is available for further processing. While not expressly illustrated, in other embodiments the first PHA phase and the second PHA phase from the system and process illustrated in FIG. 5 may be mixed to form a combined PHA phase that is available for further processing.

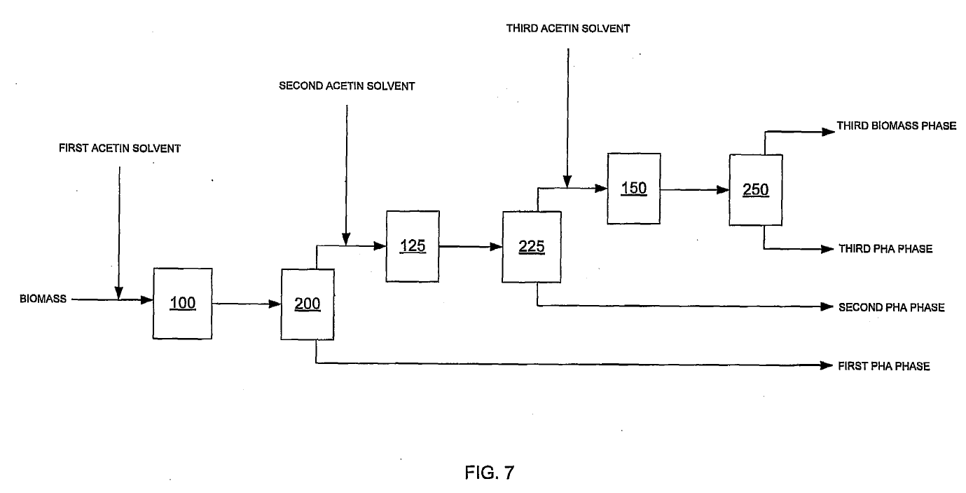

FIG. 7 is a process diagram that illustrates a non-limiting embodiment of a system and process for recovery of PHA from biomass having three disruption steps and three separation steps where a first amount of an acetin solvent is mixed with the biomass prior to the first disruption step, a second amount of an acetin solvent is mixed with the biomass prior to the second disruption step, and a third amount of an acetin solvent is mixed with the biomass prior to the third disruption step, as follows. Initially, the biomass is mixed with a first amount of acetin solvent to form a first extraction mixture. The first extraction mixture comprises at least a first PHA phase and a first biomass phase. The first PHA phase comprises at least a portion of the PHA originally present in the biomass. The first extraction mixture is introduced to the first disruption unit 100, where at least one cell in the biomass is disrupted. The first extraction mixture exits the first disruption unit 100 and is introduced to the first separation unit 200, where the first PHA phase is separated from the first biomass phase. The separated first biomass phase and first PHA phase exit the first separation unit 200. The separated first PHA phase is available for further processing.

The separated first biomass phase is mixed with a second amount of an acetin solvent (which may be the same or different in composition and/or quantity of the first amount of acetin solvent) to form a second extraction mixture. The second extraction mixture comprises at least a second PHA phase and a second biomass phase. The second PHA phase comprises at least a portion of the PHA remaining in the disrupted first biomass phase. The second extraction mixture is introduced to the second disruption unit 125, where the biomass is further disrupted. The second extraction mixture exits the second disruption unit 125 and is introduced to the second separation unit 225, where the second PHA phase is separated from the second biomass phase. The separated second biomass phase and second PHA phase exit the second separation unit 225. The separated second PHA phase is available for further processing.

The separated second biomass phase is mixed with a third amount of an acetin solvent (which may be the same or different from the first and/or second amount of acetin solvent) to form a third extraction mixture. The third extraction mixture comprises at least a third PHA phase and a third biomass phase. The third PHA phase comprises at least a portion of the PHA remaining in the disrupted second biomass phase. The third extraction mixture is introduced to the third disruption unit 150, where the biomass is further disrupted. The third extraction mixture exits the third disruption unit 150 and is introduced to the third separation unit 250, where the third PHA phase is separated from the third biomass phase. The separated third biomass phase and third PHA phase exit the third separation unit 250, and are individually available for further processing.

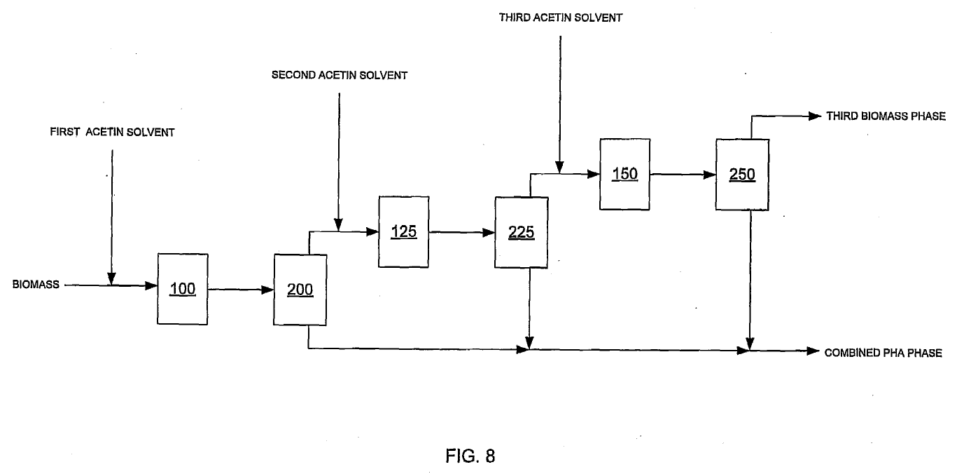

FIG. 8 is a process diagram that illustrates a non-limiting embodiment of a system and process for recovery of PHA from biomass analogous to the system and process illustrated in FIG. 7, except that the first PHA phase, the second PHA phase and the third PHA phase are mixed to form a combined PHA phase comprising at least a portion of the PHA from the biomass. The combined PHA phase is available for further processing. While not expressly illustrated, in various embodiments the biomass and the first amount of an acetin solvent, the first biomass phase and the second amount of an acetin solvent, and the second biomass phase and the third amount of an acetin solvent may be mixed subsequent to, or simultaneous with, disruption in any of disruption units 100, 125, or 150. The processes and systems described herein are not limited in this context.

In addition, the mixing of the biomass (or biomass phase) and the amount of an acetin solvent may occur prior to, simultaneous with, or subsequent to the disruption step for, any disruption/separation unit grouping within a recovery operation. For example, in a given recovery operation, the first amount of an acetin solvent may be mixed with the biomass prior to, simultaneous with, or subsequent to the first disruption step, the second amount of acetin solvent may be mixed with the first biomass phase prior to, simultaneous with, or subsequent to the second disruption step, and the third amount of acetin solvent may be mixed with the second biomass phase prior to, simultaneous with, or subsequent to the third disruption step. In certain non-limiting embodiments, the timing of the mixing of the biomass (or biomass phase) with the acetin solvent may differ according to the individual disruption steps. The processes and systems described herein are not limited in this context.

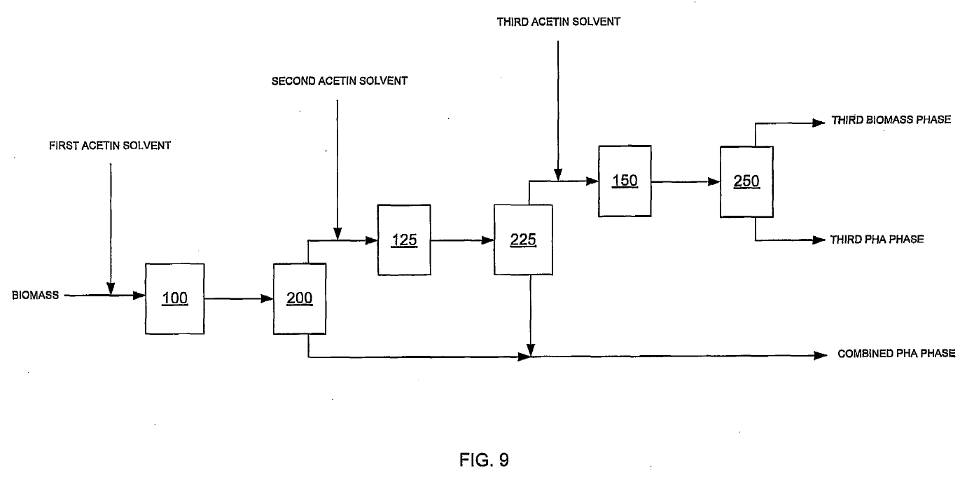

The processes and systems described herein are not limited in terms of the manner of combination, if any, of the PHA phases. For example, referring to FIG. 9, a process diagram is provided that illustrates a system and process for recovery of PHA from biomass analogous to the system and process illustrated in FIG. 8, except that only the first PHA phase and the second PHA phase are mixed to form a combined PHA phase, and the third PHA phase is available for processing separate from the processing of the combined PHA phase. In other embodiments, the first PHA phase and the third PHA phase or the second PHA phase and the third PHA phase may be combined.

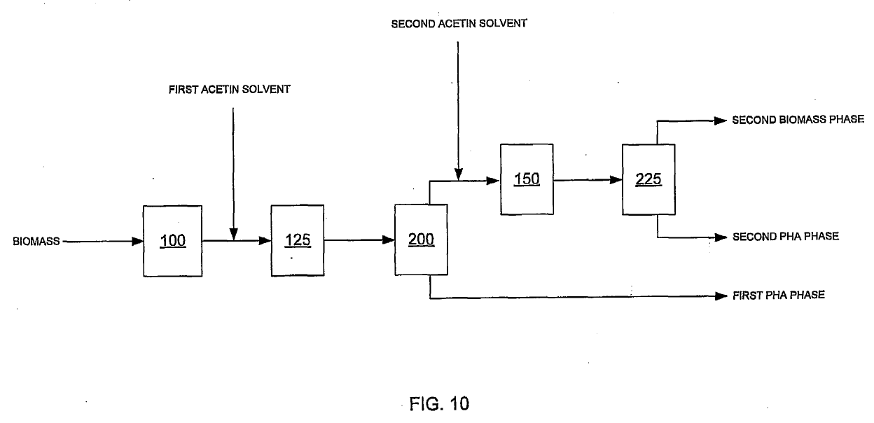

FIG. 10 is a process diagram that illustrates a non-limiting embodiment of a system and process for recovery of PHA from biomass having three disruption steps and two separation steps where a first amount of an acetin solvent is mixed with the biomass between the first disruption step and the second disruption step, and a second amount of an acetin solvent is mixed with the biomass prior to the third disruption step. The biomass is introduced to the first disruption unit 100, where at least one cell in the biomass is disrupted. The disrupted biomass exits the first disruption unit 100 and is mixed with a first amount of an acetin solvent to form a first extraction mixture. The first extraction mixture comprises at least a first PHA phase and a first biomass phase. The first PHA phase comprises at least a portion of the PHA originally present in the biomass. The first extraction mixture is introduced to the second disruption unit 125, where the biomass in the first extraction mixture is further disrupted. The first extraction mixture exits the second disruption unit 125 and is introduced to first separation unit 200, where the first PHA phase is separated from the first biomass phase. The separated first biomass phase and first PHA phase exit the first separation unit 200. The first PHA phase is available for further processing.

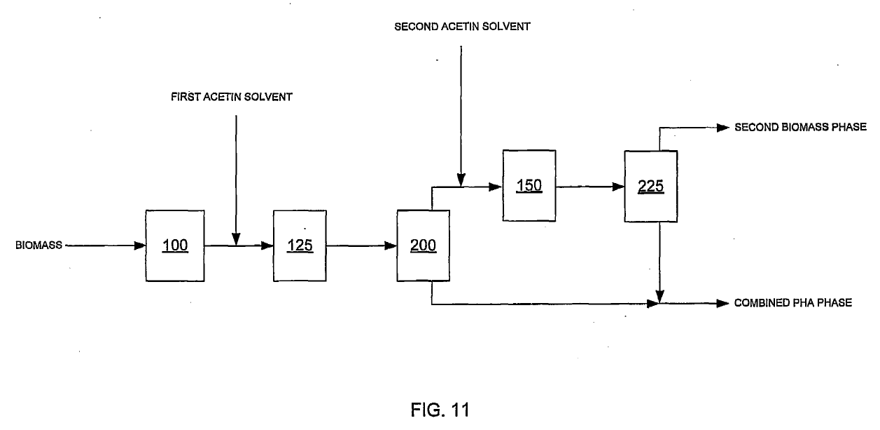

The separated first biomass phase is mixed with a second amount of an acetin solvent (which may be the same as or different from the first amount of acetin solvent) to form a second extraction mixture. The second extraction mixture comprises at least a second PHA phase and a second biomass phase. The second PHA phase comprises at least a portion of the PHA remaining in the first biomass phase. The second extraction mixture is introduced to the third disruption unit 150, where the biomass is further disrupted. The second extraction mixture exits the third disruption unit 150 and is introduced to the second separation unit 225, where the second PHA phase is separated from the second biomass phase. The separated second biomass phase and second PHA phase exit the second separation unit 225 and are individually available for further processing. In various embodiments, the first PHA phase and the second PHA phase may be mixed to form a combined PHA phase (see FIG. 11).

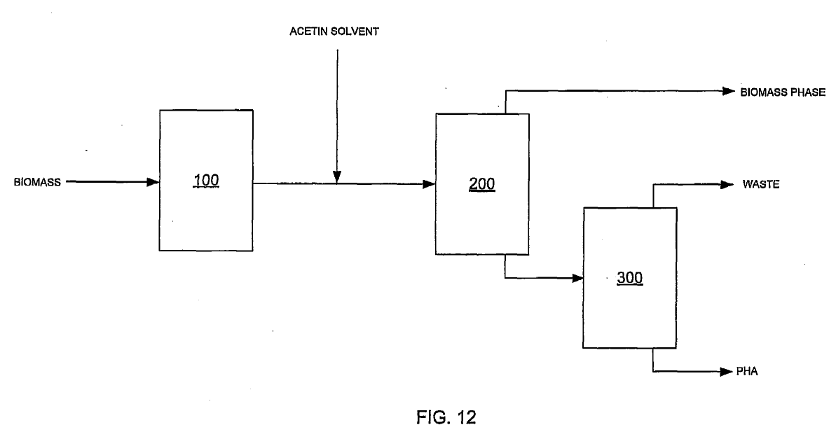

FIG. 12 is a process diagram that illustrates a non-limiting embodiment of a system and process for recovery of PHA from biomass having a disruption step, a separation step and an isolation/purification step. The biomass is introduced to disruption unit 100, where at least one cell in the biomass is disrupted. The disrupted biomass exits the disruption unit 100 and is mixed with an amount of an acetin solvent to form an extraction mixture. The extraction mixture comprises at least a PHA phase and a biomass phase. The PHA phase comprises at least a portion of the PHA originally present in the biomass. The extraction mixture is introduced to separation unit 200, where the PHA phase is separated from the biomass phase. The separated biomass phase and PHA phase exit the separation unit 200. The PHA phase is introduced to an isolation/purification unit 300, where at least a portion of the PHA is isolated from the PHA phase and/or purified. The isolation/purification unit 300 comprises at least one isolation/purification apparatus which conducts the isolation/purification operation. The isolation/purification operation may be conducted according to any suitable isolation/purification method known in the art including, for example, crystallization, precipitation, solvent displacement by a co-solvent, membrane separation, ultrafiltration, vacuum drying, short path stripping, wiped film evaporation, wiped film stripping, horizontal film rotary evaporation, horizontal film rotary stripping, centrifugation, supercritical extraction, and combinations of any two or more thereof. Isolation/purification apparatuses suitable for use in the processes and systems described herein include, for example, wiped film evaporators, short path distillation apparatuses, ultracentrifuges, and horizontal film rotary evaporators (e.g., a Rototherm™ evaporator, available from Artisan Industries, Inc., Waltham, Mass., USA).

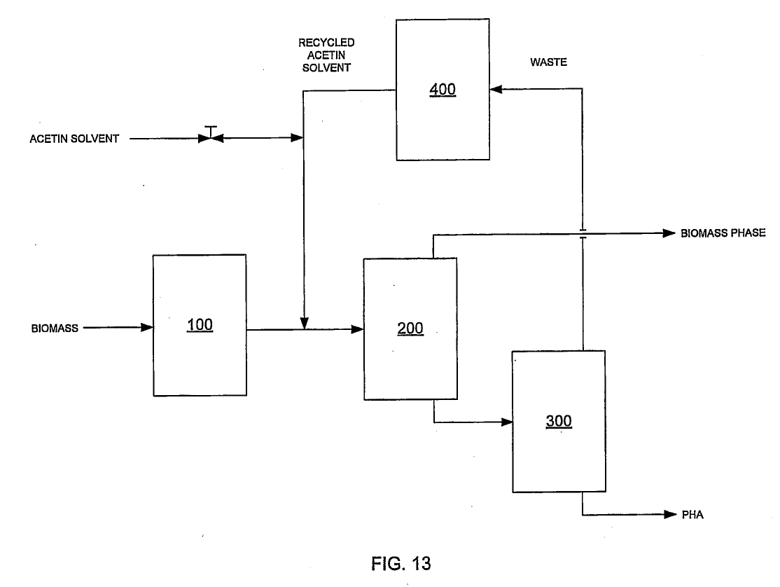

An isolated/purified PHA and waste material individually exit the isolation/purification unit 300. The isolated/purified PHA product is available for post-processing. For example, the PHA product may be subject to additional processing operations, for example, but not limited to, recrystallization, pelletization, grinding, extrusion, blow molding, injection molding, thermoforming, calendaring, spinning, casting, compression molding, transfer molding, or combinations of any thereof. The waste material may comprise acetin solvent and/or other extracted biomass components. The waste material may comprise additional materials of interest and therefore may be further processed such that those materials are isolated and purified. The waste material may also be recycled (see FIG. 13) such that waste is taken from the isolation/purification unit 300, introduced to a recycle unit 400, optionally reprocessed to an acceptable purity level (for example, by a solvent reclamation process), and mixed with fresh biomass feedstock or combined with fresh acetin solvent.

In various embodiments, the recycle unit 400 may comprise at least one acetin recycle apparatus which conducts the recycle operation. The recycle operation may be conducted according to any suitable acetin solvent recycle method (e.g., a acetin solvent reclamation process). In other embodiments, the waste may be taken from the isolation/purification unit 300 and mixed directly with fresh biomass feedstock or disrupted biomass without reclamation. In yet other embodiments, waste may be taken from the isolation/purification unit 300 and mixed with fresh acetin solvent in predetermined proportions prior to mixing with a fresh biomass feedstock or disrupted biomass.

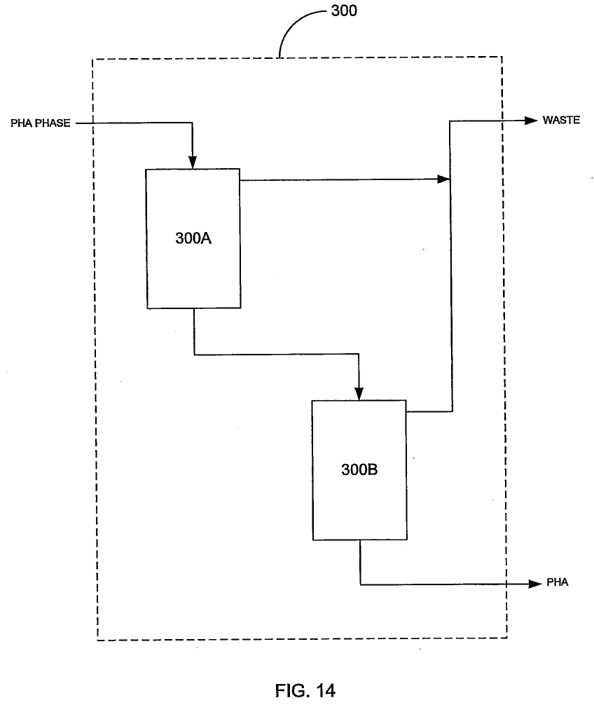

FIG. 14 is a process diagram that illustrates a non-limiting embodiment of an isolation/purification unit 300 including a wiped film evaporation unit 300A and a short path distillation unit 300B. The feed to isolation/purification unit 300 may be any of a first PHA phase, a second PHA phase, a third PHA phase, and various mixtures thereof. The PHA phase may be introduced to wiped film evaporator 300A, where an intermediate PHA fraction and a waste fraction may be formed. The intermediate PHA fraction exits the wiped film evaporator 300A and may be introduced to short path distillation apparatus 300B, where an isolated/purified PHA product and at least one waste fraction may be formed and separated. The waste fractions from the wiped film evaporator 300A and the short path distillation apparatus 300B may be combined to form the waste material, which may comprise waste acetin solvent. The isolated/purified PHA product is available for further post-recovery processing. Other various combinations of isolation techniques are also contemplated.

### EXAMPLE 1

Samples of PHA are tested for solubility in triacetin solvent. A small amount of PHA dissolves in triacetin at room temperature. Temperature is gradually increased and more substantial amounts of PHA dissolve beginning at about 50° C. It is determined that triacetin solvent at 80° C. will dissolve PHA so as to form a solution of up to 6% PHA by weight. The solution of 6% PHA in triacetin is observed as clear and more viscous than pure triacetin solvent, and is readily pourable. The PHA is increasingly soluble in triacetin solvents as solvent temperature increases. The solubility of the PHA in triacetin also increases with decreasing PHA particle size. Therefore, increasing the degree of pulverization of the PHA samples increases the solubility of the PHA in triacetin. A solvent mixture of triacetin and certain other acetins improves the solubility of the PHA in the solvent, perhaps due to increased polarity of the solvent mixture.

### EXAMPLE 2

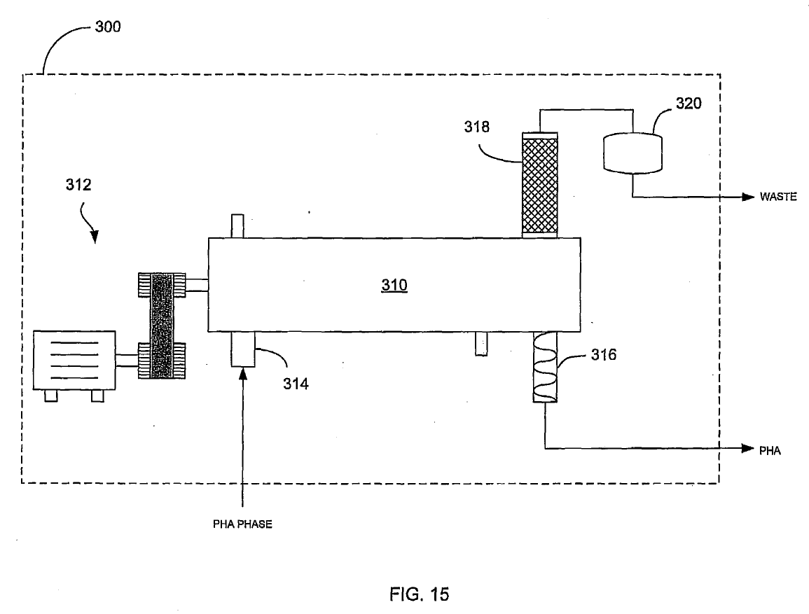

A quantity of biomass containing PHA is disrupted in a ball mill. The disrupted biomass is contacted with an acetin solvent to form an extraction mixture. The extraction mixture includes residual biomass and the acetin solvent. A portion of the PHA dissolves from the biomass in the acetin solvent, forming a PHA phase. The residual biomass is separated from the acetin solvent (PHA phase). The acetin solvent is introduced to an isolation/purification unit. FIG. 15 is a schematic that illustrates the isolation/purification unit (designated 300). The isolation/purification unit 300 includes a Rototherm™ evaporator 310 (available from Artisan Industries, Inc., Waltham, Mass., USA). The evaporator 310 comprises a drive assembly 312, a feed inlet 314, a product discharge 316, a solvent vapor outlet column 318, and a vacuum receiver 320. The PHA phase is fed to the evaporator 310 through inlet 314. The drive assembly 312 drives the internal rotor blades (not shown) which impart a centrifugal force to the PHA phase introduced to the evaporator 310 by way of feed inlet 314. The centrifugal force exerted by the rotor blades holds a thin film of the PHA phase against the heated process wall (not shown). The thin film between the rotor blades and the process wall continuously covers a heated section of the process wall, and while thin the film is constantly renewed as the progressively more concentrated PHA fraction is displaced towards the product discharge 316 by the incoming PHA phase feed. The waste vapor fraction is drawn up through the solvent vapor outlet column 318 and the vacuum receiver 320 to form the waste material, which comprises waste acetin solvent. The isolated/purified PHA product is removed from the system through the product discharge 316. The product discharge 316 includes an auger to assist in the removal of the solid and/or molten PHA. The isolated/purified PHA product is available for any further post-recovery processing.

Although various embodiments have been described herein, many modifications and variations to those embodiments may be implemented. For example, different types of biomass disruption equipment, phase separation equipment, PHA isolation/purification equipment, and solvent recycle equipment may be employed in any given unit operation. In addition, the order, sequence and number of unit operations and/or processing steps may be varied. For example, acetin solvent may be mixed with biomass feedstock and/or separated biomass phases prior to, subsequent to, and/or simultaneous with disruption of the biomass. Any given processing unit may comprise any number of individual unit operations performed by any number of processing devices/apparatuses. Furthermore, embodiments have been described involving one, two and three disruption and/or separation steps. However, the systems and processes described herein are not limited to any particular number of disruption and/or separation steps, and may involve any number of unit operations organized in a serial, parallel or combined manner.

Moreover, the systems and processes described herein may be operated in a batch, semi-batch or continuous mode. The specific devices and apparatuses utilized to implement the systems and processes described herein, and the manner of material transport within and between the various unit operations, may be selected as suitable according to material processing principles known in the art. The foregoing description and following claims are intended to cover all modifications and variations.

We claim:

1. A process for recovery of polyhydroxyalkanoate from biomass comprising:
extracting at least a portion of polyhydroxyalkanoate from a quantity of biomass with an acetin solvent to provide a biomass phase and a polyhydroxyalkanoate phase comprising the at least a portion of the polyhydroxyalkanoate; and
separating the polyhydroxyalkanoate phase from the biomass phase.

2. The process of claim 1, wherein the acetin solvent is selected from the group consisting of triacetin, 1,2-diacetin, 1,3-diacetin, 1-monoacetin, 2-monoacetin, and mixtures of any two or more thereof.

3. The process of claim 1, wherein extracting at least a portion of polyhydroxyalkanoate from a quantity of biomass with an acetin solvent comprises contacting the biomass and the acetin solvent, wherein the acetin solvent is at a temperature from about 25° C. to about 200° C.

4. The process of claim 1, wherein the biomass is selected from the group consisting of a microorganism capable of producing polyhydroxyalkanoate and an agricultural biomass capable of producing polyhydroxyalkanoate.

5. The process of claim 1, wherein the biomass comprises an agricultural crop capable of producing polyhydroxyalkanoate, wherein the crop is selected from the group consisting of corn stover, a switchgrass, a sugarcane, an oil seed, and combinations of any two or more thereof.

6. The process of claim 1, wherein the biomass is a genetically modified microorganism capable of producing polyhydroxyalkanoate.

7. The process of claim 1, wherein from about 0.1% to about 20% of the polyhydroxyalkanoate phase by weight is polyhydroxyalkanoate.

8. The process of claim 1, further comprising:
disrupting one or more cells in the biomass.

9. The process of claim 8, wherein disrupting one or more cells in the biomass comprises at least one of pulverizing one or more cells of the biomass and micropulverizing one or more cells of the biomass.

10. The process of claim 8, wherein disrupting one or more cells in the biomass is performed in a disrupting device selected from the group consisting of a ball mill, a pulverizing mill, a hammer mill, a cell grinder, an ultrasonicator, and combinations of any two or more thereof.

11. The process of claim 1, wherein separating the polyhydroxyalkanoate phase from the biomass phase comprises a process selected from the group consisting of settling, centrifuging, filtering, decanting, and combinations of any two or more thereof.

12. The process of claim 1, further comprising:
isolating the at least a portion of the polyhydroxyalkanoate from the polyhydroxyalkanoate phase.

13. The process of claim 12, wherein isolating the at least a portion of the polyhydroxyalkanoate from the polyhydroxyalkanoate phase comprises at least one of cooling the polyhydroxyalkanoate phase, adding a co-solvent to the polyhydroxyalkanoate phase, and removing at least a portion of the acetin solvent.

14. The process of claim 12, comprising purifying the at least a portion of the polyhydroxyalkanoate using a process selected from the group consisting of membrane separation, ultra filtration, vacuum drying, short path stripping, wiped film evaporation, wiped film stripping, horizontal film rotary evaporation, horizontal film rotary stripping, centrifuging, supercritical extraction, and combinations of any thereof.

15. A process for the recovery of polyhydroxyalkanoate from biomass comprising:
disrupting one or more cells in a quantity of biomass comprising polyhydroxyalkanoate;
extracting at least a first portion of the polyhydroxyalkanoate from the biomass with a first amount of an acetin solvent to provide a first biomass phase and a first polyhydroxyalkanoate phase comprising the at least a first portion of the polyhydroxyalkanoate; and
separating the first polyhydroxyalkanoate phase from the first biomass phase.

16. The process of claim 15, further comprising:
contacting the first biomass phase with a second amount of an acetin solvent;
disrupting one or more cells in the first biomass phase to provide a second biomass phase and a second polyhydroxyalkanoate phase comprising at least a second portion of a polyhydroxyalkanoate; and
separating the second polyhydroxyalkanoate phase from the second biomass phase.

17. The process of claim 16, further comprising:
combining the first polyhydroxyalkanoate phase with the second polyhydroxyalkanoate phase to provide a combined polyhydroxyalkanoate phase; and
isolating the polyhydroxyalkanoate from the combined polyhydroxyalkanoate phase.

18. The process of claim 15, wherein the biomass and the first amount of acetin solvent are mixed prior to disrupting one or more cells of the biomass.

19. The process of claim 15, wherein the acetin solvent is selected from the group consisting of triacetin, 1,2-diacetin, 1,3-diacetin, 1-monoacetin, 2-monoacetin, and mixtures of any two or more thereof.

20. The process of claim 15, wherein the first amount of the acetin solvent is at a temperature from about 25° C. to about 200° C.

21. The process of claim 15, wherein from about 0.1% to about 20% of the first polyhydroxyalkanoate phase by weight is polyhydroxyalkanoate.

22. A process for the recovery of polyhydroxyalkanoate from biomass comprising:
disrupting one or more cells in a quantity of biomass comprising polyhydroxyalkanoate;
extracting with a first amount of an acetin solvent at a temperature from about 25° C. to about 200° C. at least a portion of the polyhydroxyalkanoate from the biomass to provide a first biomass phase and a first polyhydroxyalkanoate phase comprising a first portion of PHA, wherein the first PHA phase comprises from about 0.1% to about 20% by weight of polyhydroxyalkanoate, wherein the acetin solvent is selected from the group consisting of triacetin, 1,2-diacetin, 1,3-diacetin, 1-monoacetin, 2-monoacetin, and mixtures of any two or more thereof;
separating the first polyhydroxyalkanoate phase from the first biomass phase; and
isolating at least a portion of the polyhydroxyalkanoate from the first polyhydroxyalkanoate phase.

23. The process of claim 22, further comprising:
contacting the first biomass phase with a second amount of an acetin solvent;
disrupting one or more cells in the first biomass phase to provide a second biomass phase and a second polyhydroxyalkanoate phase comprising at least a second portion of polyhydroxyalkanoate;
separating the second polyhydroxyalkanoate phase from the second biomass phase; and
isolating at least a portion of polyhydroxyalkanoate from the second polyhydroxyalkanoate phase.

24. The process of claim 23, wherein isolating at least a portion of the polyhydroxyalkanoate from the second polyhydroxyalkanoate phase comprises:
combining the second polyhydroxyalkanoate phase with the first polyhydroxyalkanoate phase to provide a combined polyhydroxyalkanoate phase; and
isolating at least a portion of the polyhydroxyalkanoate from the combined polyhydroxyalkanoate phase.

25. The process of claim 23, comprising:
contacting the second biomass phase with a third amount of an acetin solvent;
disrupting one or more cells in the second biomass phase to provide a third biomass phase and a third polyhydroxyalkanoate phase comprising at least a third portion of polyhydroxyalkanoate;
separating the third polyhydroxyalkanoate phase from the third biomass phase; and
isolating at least a portion of polyhydroxyalkanoate from the third polyhydroxyalkanoate phase.

26. A system for recovery of polyhydroxyalkanoate from biomass comprising:
at least one disruption unit comprising at least one cell disruption device; and
at least one separation unit comprising at least one separation apparatus,
wherein the disruption unit functions to disrupt at least one cell in a quantity of biomass, and wherein the separation unit functions to separate a PHA phase from a biomass phase, wherein the PHA phase and the biomass phase are formed after the quantity of biomass is contacted with an amount of an acetin solvent.
